(19) United States
(12) Patent Application Publication
(10) Pub. No.: US 2003/0181861 A1
(43) Pub. Date: Sep. 25, 2003

(54) Safety device

(75) Inventor: Bradley M. Wilkinson, North Haledon, NJ (US)

Correspondence Address: CASELLA & HESPOS LLP, 274 MADISON AVENUE, SUITE 1703, NEW YORK, NY, 10016-0701, US

(73) Assignee: Becton, Dickinson and Company, Franklin Lakes, NJ

(21) Appl. No.: 10/392,255

(22) Filed: Mar. 17, 2003

Related U.S. Application Data
(60) Provisional application No. 60/366,368, filed on Mar. 20, 2002.

(51) Int. Cl. A61M 5/32

(52) U.S. Cl. 604/192

(57) ABSTRACT

A safety assembly includes a holder with a proximal end, a distal end and a tubular sidewall extending between the ends. The proximal end is configured to engage a needle assembly. The distal end is configured to slidably receive a fluid collection tube. A shield is hingedly mounted to the distal end of the holder and can rotate from a first position where the shield lies adjacent the holder to a second position where the needle is shielded. A spring is disposed for biasing the shield to the second position. A latch is disposed on the holder for movement between a locking position where the latch holds the shield in the first position and a release position where the latch permits the shield to be propelled to the second position.

### RELATED APPLICATIONS

This application claims priority on U.S. Provisional Patent Appl. No. 60/366,368 filed Mar. 20, 2002.

### FIELD OF THE INVENTION

The present invention relates to a shield for a needle and more particularly to a safety shield that may be used in conjunction with a needle assembly and a needle holder.

### BACKGROUND OF THE INVENTION

Disposable medical devices having piercing elements for administering a medication or withdrawing a fluid, such as hypodermic needles, blood collecting needles, fluid handling needles and assemblies thereof, require safe and convenient handling. The piercing elements include, for example, pointed needle cannula or blunt ended cannula.

Safe and convenient handling of disposable medical devices is recognized by those in the medical arts so as to minimize exposure to blood borne pathogens. Safe and convenient handling of disposable medical devices results in the disposal of the medical devices intact.

As a result of this recognition, numerous devices have been developed for shielding needles after use. Many of these devices are somewhat complex and costly. In addition, many of these devices are cumbersome to use in performing procedures. Furthermore, some of the devices are so specific that they preclude use of the device in certain procedures or with certain devices and/or assemblies.

Therefore, there exists a need for a safety shield assembly: (i) that is manufactured easily; (ii) that is applicable to many devices; (iii) that is simple to use with one hand; (iv) that can be disposed of safely; (v) that does not interfere with normal practices of needle use; (vi) that has tactile features whereby the user may be deterred from contacting the needle, the user may easily orient the needle with the patient and easily actuate and engage the shield assembly; (vii) that has visual features whereby the user may be deterred from contacting the needle, the user may easily orient the needle with the patient and easily actuate and engage the shield assembly; (viii) that is not bulky; (ix) that includes means for minimizing exposure to the user of residual fluid leaking from the needle; and (x) provides minimal exposure to the user because the needle shield is immediately initiated by the user after. the needle is withdrawn from the patient's vein.

### SUMMARY OF THE INVENTION

The present invention is a safety shield assembly that comprises: a shield; means for connecting the shield to a fluid handling device that contains a piercing element, such as needle; means for securely covering and/or containing the needle within the shield and means for securely locking the shield in a final non-retractable closed position over the needle.

Preferably, the shield comprises a rearward end, a forward end, a slot or longitudinal opening for housing the used needle in the forward end, means for securing the needle in the slot, means for guiding the needle into the slot, means for connecting the shield and the fluid handling device, means for guiding the user's fingers to move the shield into various positions, and means for retaining the shield securely over the used needle.

The shield may be connected to the fluid handling device by a hanger bar that engages with a hook arm on the fluid handling device so that the shield may be pivoted with respect to the fluid handling device into several positions. It is within the purview of the present invention to include any structure for connecting the shield to the fluid handling device so that the shield may be pivoted with respect to the fluid handling device. These structures include known mechanical hinges and various linkages, living hinges, or combinations of hinges and linkages.

The shield may be connected to the fluid handling device by an interference fit between the hanger bar and the hook arm. Therefore, the shield always is oriented in a stable position and will not move forward or backwards unless movement of the shield relative to the hanger bar and the hook bar is initiated by the user.

Alternatively, the shield and fluid handling device may be a unitary one-piece structure. The one-piece structure may be obtained by many methods, including molding the shield and the fluid handling device as a one-piece unit, thereby eliminating the separate shield and the fluid handling device during the manufacturing assembly process.

The assembly of the present invention may further comprise tactile and visual means for deterring the user from contacting the needle, providing easy orientation of the needle with the patient and providing the user with a guide for actuation and engagement with the shield.

The assembly of the present invention may further comprise means for minimizing exposure by the user to residual fluid leaking from a used needle. For example, a polymer material, such as a gel, may be located in the shield.

Most desirably, the assembly of the present invention is such that the cooperating parts of the assembly provide the means for the shield to move into a forward position over the needle. Thus, by simple movement of the shield into a forward position over the used needle, the assembly is ready for subsequent disposal. Therefore, the safety shield assembly of the present invention provides minimal exposure of the user to a needle because the shielding is initiated by the user immediately after the needle is withdrawn from the patient's vein.

Desirably, the assembly of the present invention may be used with a syringe assembly, a hypodermic needle, a needle assembly, a needle assembly with a needle holder, a blood collection set, an intravenous infusion set or other fluid handling devices. Preferably, the assembly of the present invention is used with a needle assembly comprising a needle and a hub. Preferably the needle is a conventional double ended needle.

Most preferably, the present invention is used with a needle assembly comprising a hub and a needle connected to the hub whereby the needle comprises a non-patient end and an intravenous end. The fluid handling device of the present invention may comprise a hook arm and the shield may be connected movably to the hook arm. Thus the shield may be position with respect to the fluid handling device and moved easily into several positions.

Preferably, the fluid handling device is fitted non-rotatably with the hub of the needle assembly. Additionally, the fluid handling device includes cooperating means that mate with reciprocal means on the shield to help retain the shield in a final closed position, to propel the shield toward the final closed portion and to provide a clear audible and tactile indication of complete shielding.

The shield preferably includes a cannula finger lock for locked engagement with the cannula when the shield is in the final closed position around the needle cannula. The cannula finger lock preferably projects obliquely from one sidewall of the shield angularly toward the opposed sidewall and the top wall of the shield. The cannula finger lock is dimensioned, disposed and aligned to contact the needle cannula when the shield approaches the final closed position. Contact between the cannula and the cannula finger lock will cause the cannula finger lock to resiliently deflect toward the sidewall from which the cannula finger lock extends. Sufficient rotation of the shield will cause the needle cannula to pass the cannula finger lock. As a result, the cannula finger lock will resiliently return to or toward its undeflected condition for securely trapping the needle cannula in the shield.

Preferably, the fluid handling device is fitted with the hub of the needle assembly so that the fluid handling device cannot rotate around the hub. Additionally, the fluid handling device includes cooperating means that mate with reciprocal means on the shield to propel the shield toward a final closed position.

Alternatively, the fluid handling device and hub may be a unitary one-piece structure. The one piece structure may be accomplished by many methods including molding the fluid handling device and the hub as a one-piece unit thereby eliminating the need to separately assemble the fluid handling device to the hub during the manufacturing process.

Most preferably, the fluid handling device is fitted with the hub of the needle assembly so that the bevel surface or bevel up surface of the intravenous or distal end of the needle faces the same side of the fluid handling device when the shield is in the open position. Alignment of the fluid handling device, hub, shield and needle with the bevel surface up makes it easier to insert the needle into the patient without manipulating the assembly. The orientation of the intravenous end of the needle with the bevel up assures the user that the needle is oriented properly for use and does not require any manipulation before use. Most notably, the orientation of the shield provides a visual indication to the user of the orientation of the bevel surface of the needle.

Alternatively, it is within the purview of the present invention that the shield, fluid handling device and hub is a unitary one-piece structure. The one-piece structure may be accomplished by many methods including molding the shield, fluid handling device and hub as a one piece unit thereby eliminating the need to separately assemble the shield, fluid handling device and hub during the manufacturing process.

Another notable attribute of the present invention is that the tactile and visual features deter the user from touching the needle, allow the user to easily orient the needle with the patient and guide the user to actuate and engage the shield of the assembly.

### BRIEF DESCRIPTION OF THE DRAWINGS

FIG. 1 is an exploded perspective view of a shield, needle holder and needle assembly in accordance with the first embodiment of the invention.

FIG. 2 is an exploded perspective view of a prior art needle assembly for use with the holder of FIG. 1.

FIG. 3 is a longitudinal cross-sectional view showing the shield in the open condition with the needle cannula in the exposed ready-to-use position.

FIG. 4 is a cross-sectional view similar to FIG. 3, but showing the shield in the shielded position.

FIG. 5 is a cross sectional view similar to FIG. 3, but showing a second embodiment with a rotatable flange for releasing the shield.

FIG. 6 is an end elevational view of the embodiments shown in FIG. 5.

FIG. 7 is a perspective view similar to FIG. 1, showing a third embodiment with a rotatable lock for holding the shield in the open condition.

FIG. 8 is a cross-sectional view taken along line 8-8 in FIG. 7.

FIG. 9 is a perspective view similar to FIG. 7, but showing a fourth embodiment of a lock for holding the shield in the open position.

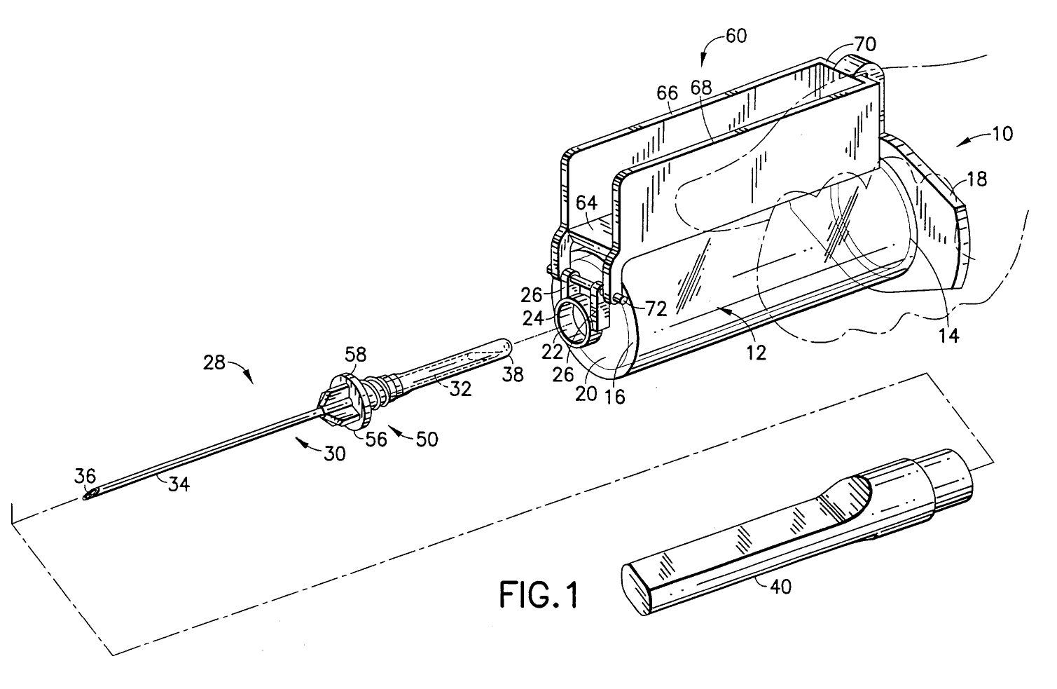

FIG. 1O is a perspective view of a fifth embodiment.

### DETAILED DESCRIPTION OF THE PREFERRED EMBODIMENTS

While this invention is satisfied by embodiments in many different forms, there is shown in the drawings and will herein be described in detail, the preferred embodiments of the invention, with the understanding that the present disclosure is to be considered as exemplary of the principles of the invention and is not intended to limit the invention to the embodiments illustrated. Various other modifications will be apparent to and readily made by those skilled in the art without departing from the scope and spirit of the invention. The scope of the invention will be measured by the appended claims and their equivalents.

A holder in accordance with the subject invention is identified generally by the numeral 10 in FIG. 1. Holder 10 preferably is formed unitarily from a plastic material and includes a tubular side wall 12 with a proximal end 14 and a distal end 16. Proximal end 14 of tubular side wall 12 is widely open and is dimensioned to slidably accommodate an evacuated blood collection tube (not shown). A flange 18 projects outwardly at proximal end 14 of tubular side wall 12 to facilitate gripping and manipulation of holder 10.

An annular distal end wall 20 extends inwardly at distal end 16 of tubular side wall 12. A central aperture 22 extends through distal end wall 20 and communicates with internal portions of holder 10. Holder 10 is further characterized by an internally threaded cylindrical collar 24 formed concentrically with aperture 22. Additionally, hook arms 26 extend distally from distal end wall 20.

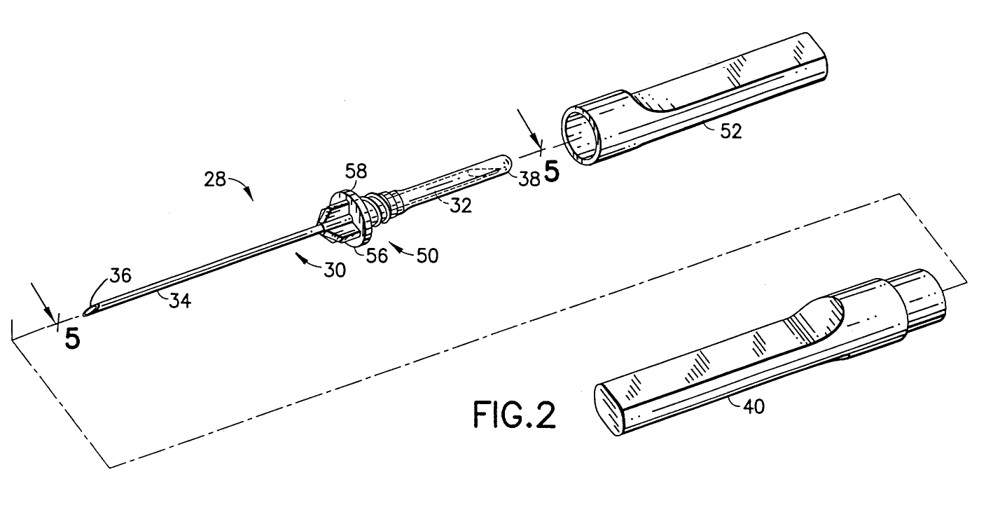

Holder 10 is employed with a known needle assembly 28. Needle assembly 28 includes a needle 30 with a non-patient end 32, an intravenous end 34 and a passageway 36 extending between the non-patient end and the intravenous end. An elastomeric sleeve 38 covers the non-patient end. A first rigid sleeve 40 covers the intravenous end and a second rigid sleeve 42 covers the non-patient end and the elastomeric sleeve, as shown in FIG. 2.

Needle assembly 28 further includes a hub 50 with a threaded end 54, a ribbed end 56 and a passageway extending between the threaded end and the ribbed end. Threaded end 54 and ribbed end 56 are separated by a flange 58. Non-patient end 32 of needle 30 extends from threaded end 54, and intravenous end 34 of needle 30 extends from ribbed end 56. Threaded end 54 comprises male threads for mounting of 50 in internally threaded collar 24 of holder 10. In the mounted condition, non-patient end 32 of needle 30 projects into the area bounded by cylindrical side wall 12 of holder 10.

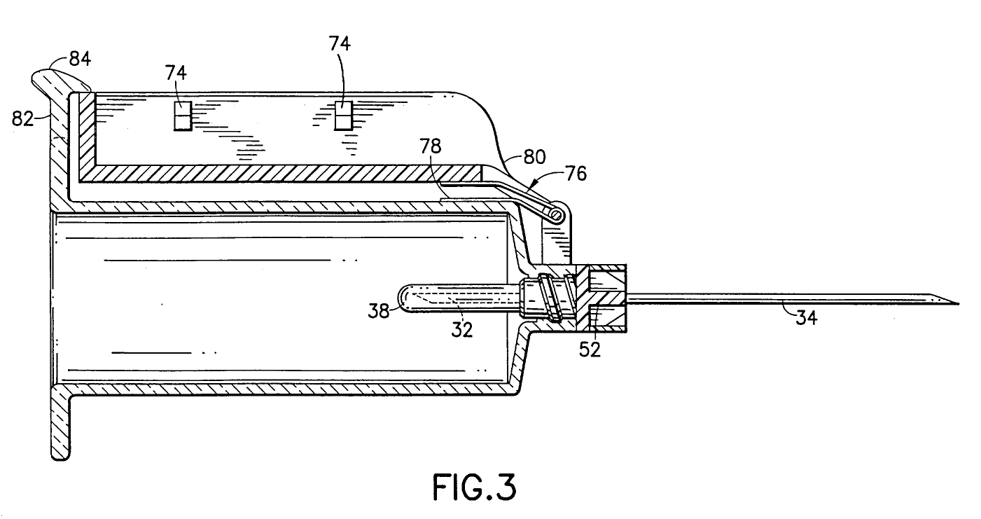

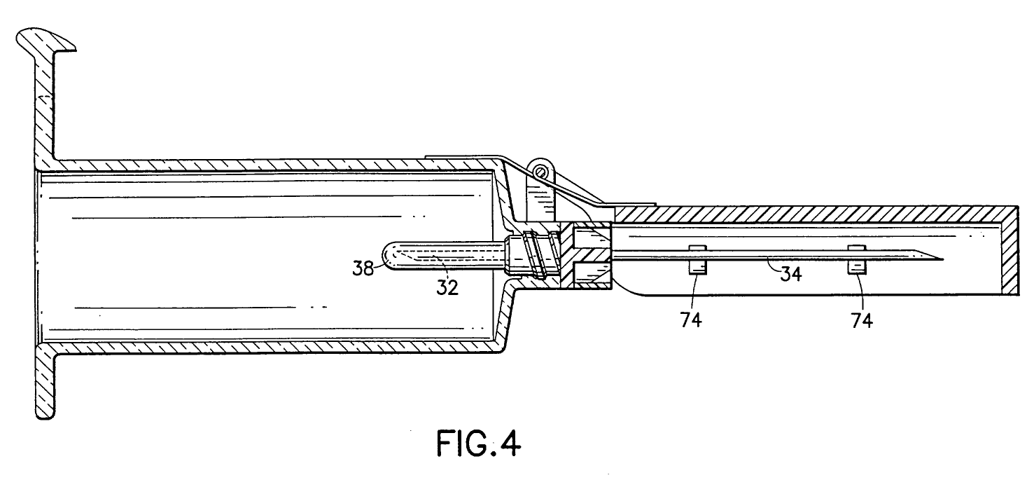

Holder assembly 10 further includes a shield 60 that is formed unitarily from a substantially rigid plastic. Shield 60 includes an elongate base wall 64, opposed side walls 66 and 68 that project from opposite sides of base wall 64 and an end wall 70 that extends from base wall 64 and between side walls 66 and 68. Hanger bar 72 projects from an end of shield 60 opposite end wall 70 and is rotatably mounted to hook arms 26. Thus, shield 60 can be rotated about hook arms 26 from an open position as shown in FIGS. 1 and 3 to a closed position as shown in FIG. 4. Shield 60 is dimensioned such that end wall 70 is substantially adjacent flange 18 of holder 10 when shield 60 is in the open position of FIGS. 1 and 3. Additionally, shield 60 is dimensioned such that end wall 70 is disposed distally of needle 30 when shield 60 is rotated to the closed position of FIG. 4. Side walls 66 and 68 of shield 60 are spaced from one another by a distance that exceeds the width of needle 30. Furthermore, side walls 66 and 68 and end wall 70 project sufficiently from base wall 64 to completely surround needle 30.

Shield 60 is formed with needle locks 74 that project from side wall 68 angularly toward base wall 64. Locks 74 are disposed and configured to contact needle 30 as shield 60 is rotated into the closed position. This contact will cause locks 74 to resiliently deflect. However, locks 74 will return to an undeflected condition and trap needle 30 after shield 60 reaches the closed position of FIG. 4.

Holder assembly 10 further includes a spring 76 mounted to or near hook arms 26. In the preferred embodiment, spring 76 is a torsion spring having a first leg 78 biased against an external surface of holder 10 and a second leg 80 biased against a surface of shield 60. Legs 78 and 80 are biased into a stored-energy position when shield 60 is in the open position of FIGS. 1 and 3. However, spring 76 is configured and disposed to bias shield 60 completely to the closed position of FIG. 4. The torsion spring, illustrated in FIGS. 1-4, can be replaced by a leaf spring mounted at substantially the same location on holder 10. Alternatively, the shield can be joined unitarily to holder 10 by an over-center living hinge. Over-center hinges on a shield are shown, for example, in U.S. Pat. No. 5,242,417.

Shield 60 is retained releasably in the open position and against the forces of spring 76 by a latch 82. Latch 82 protects unitarily from flange 18 and releasably engages an edge of end wall 70 of shield 62 spaced from base wall 64. Latch 82 is dimensioned and configured to be resiliently deflectable relative to flange 18 and includes an actuator tab 84 dimensioned and configured for convenient digital manipulation.

Holder 10 may be used substantially in a conventional manner to draw samples of blood or other bodily fluid from a patient. In particular, second rigid sleeve 42 is separated from needle assembly 28 to expose elastomeric sleeve 38. Threaded end 54 of hub 50 then is engaged threadedly in aperture 24 of holder 10. First rigid sleeve 40 then can be removed from hub 50 to expose intravenous end 34 of needle 30. Intravenous end 34 of needle 30 then can be placed in communication with a blood vessel or other source of fluid and an evacuated tube can be urged into open proximal end 14 of side wall 12. Non-patient end 42 of needle 40 thus is placed in communication with the evacuated tube for collecting specimens of blood or other bodily fluid. A plurality of evacuated tubes may be inserted sequentially into holder 10. After a sufficient volume of fluid has been collected, intravenous end 34 of needle 30 is removed from the patient. The health care worker then deflects latch 82 sufficiently to separate latch 82 from shield 60. As a result, spring 76 propels shield 60 from the open position shown in FIGS. 1 and 3 to the closed position shown in FIG. 4. Needle locks 74 engage needle 30 as shield 60 is rotated into its closed position.

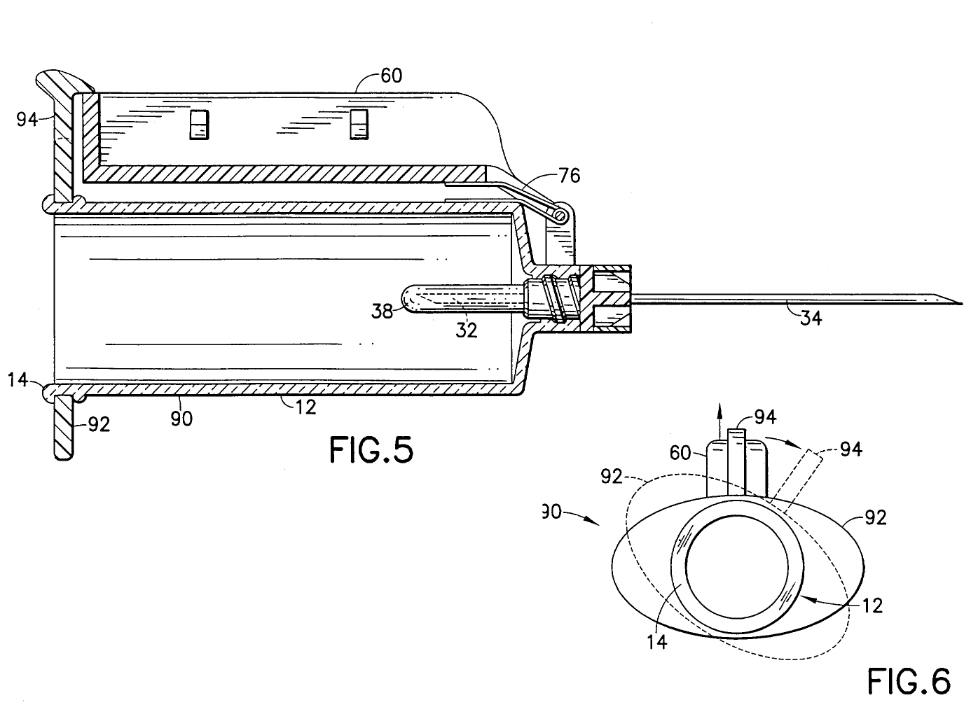

Latch 76 illustrated in FIGS. 1, 3 and 4 is just one of several optional latch configurations. In this regard, a second embodiment of the needle holder 90 is illustrated in FIGS. 5 and 6 and is substantially identical to the first embodiment of FIGS. 1, 3 and 4. However, holder 90 includes a rotatable flange 92 mounted to proximal end 14 of side wall 12. A latch 94 projects rigidly and substantially non-deflectably from flange 92. As a result, latch 94 can be separated from shield 60 merely by rotating flange 92 about proximal end 14 of side wall 12 from a first position where latch 94 engages shield 62 to a second position where latch 94 is rotatably spaced from shield 62, as shown in broken lines in FIG. 6. Thus, shield 60 will be propelled to the closed position by spring 76 after latch 94 is rotated away from shield 60.

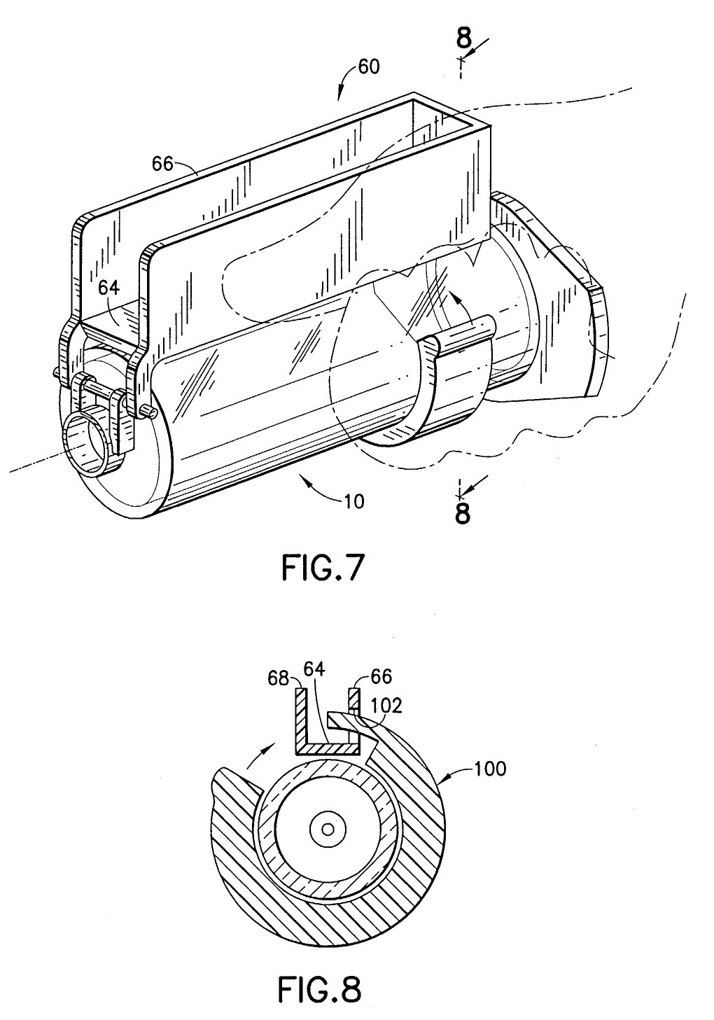

A third embodiment is illustrated in FIGS. 7 and 8, and includes a substantially C-shaped latch 100 rotatably mounted on the exterior of cylindrical side wall 12 of holder 10 at a location adjacent flange 18. In this embodiment, side wall 66 of shield 60 is formed with a slot 102 adjacent base wall 64 for receiving an end of C-shaped latch 100. Thus, C-shaped latch 100 can be rotated in the direction of the arrow shown in FIGS. 7 and 8 about the axis of holder 10 from a first position where latch 100 engages in aperture 102 of side wall 68 to a second position where latch 100 is spaced from shield 60. Thus, shield 60 will be propelled into the closed position when C-shaped latch 100 is spaced from aperture 102 in side wall 68 of shield 60.

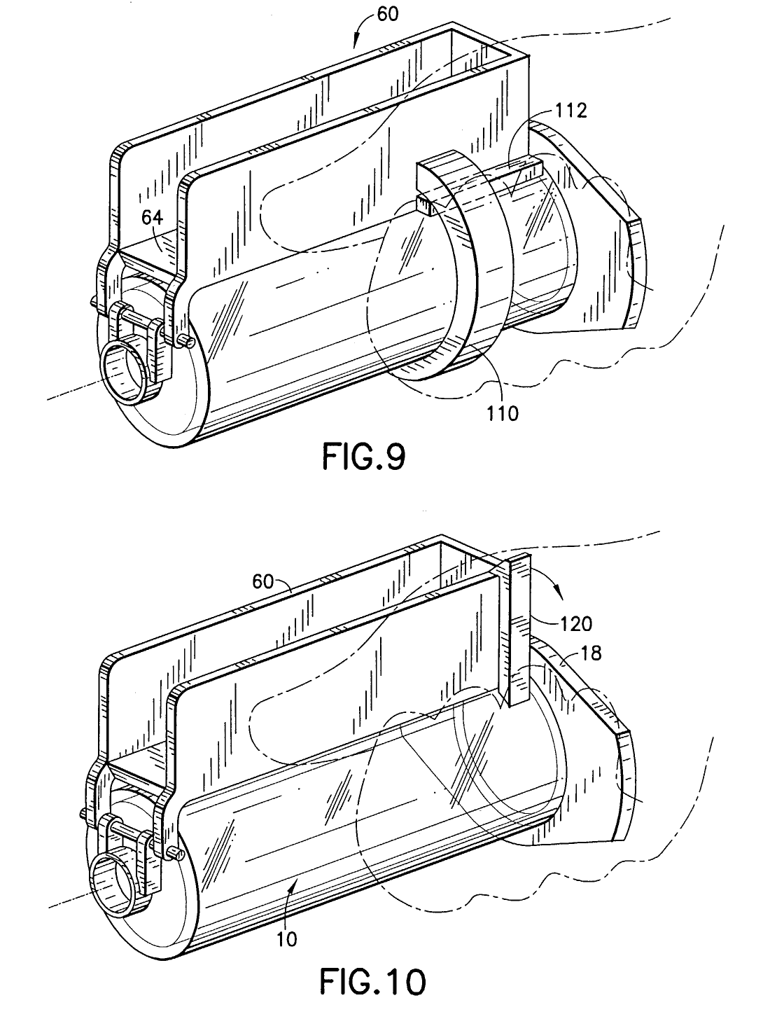

A fourth embodiment is illustrated in FIG. 9 and includes a C-shaped latch 110 that is axially slidable on the exterior of side wall 12 of holder 10. In a first range of axial movement, latch 110 engages a flange 112 that projects transversely from base wall 64 of shield 60. Thus, latch 110 prevents shield 60 from being propelled into the closed position. However, a distal movement of latch 110 relative to shield 60, as shown by the arrow in FIG. 9, separates latch 110 from shield 60 and enables shield 60 to be propelled into the closed position shown in FIG. 4.

A fifth embodiment is illustrated in FIG. 9 and includes a latch 120 that projects transversely outwardly from a portion of tubular side wall 12 of holder 10 adjacent flange 18. Latch 120, however, is disposed and configured to be deflected in the directions of the arrow in FIG. 10 about an axis that extends substantially parallel to the axis of holder 10. In other respects, however, latch 120 is functionally similar to latch 80 described above and illustrated in FIGS. 1, 3 and 4.

While the invention has been described with respect to certain preferred embodiments, it is apparent that variations can be made without departing from the scope of the invention as defined above. For example, the shield can be locked in the closed position by engagement means formed on the shield and on distal portions of the holder or on the needle hub. These locking means on the shield and on the holder or hub can be used in place of cannula latches or in addition to the above-described cannula latches.

What is claimed is:

1. A safety assembly comprising:
a holder having a proximal end, a distal end and a tubular sidewall extending between the ends, said proximal end being configured for engaging a needle;
a shield hingedly connected to said holder at a location adjacent said distal end of said holder and being rotatable from a first position where said shield lies substantially adjacent said holder to a second position where said shield extends distally beyond said holder;
a spring disposed in proximity to the hinged connection of said shield to said holder for biasing said shield to said second position; and
a latch substantially adjacent said proximal end of said holder and being movable from a locking position where said latch releasably holds said shield in said first position and a release position where said latch permits said spring to bias said shield to said second position.

2. The safety assembly of claim 1, wherein the shield in said first position extends substantially from said distal end of said holder to said proximal end thereof.

3. The safety assembly of claim 1, wherein said second position of said shield is spaced from said first position of said shield by approximately 180°.

4. The safety assembly of claim 1, wherein said shield defines a substantially U-shaped channel having an open side facing away from said holder when said shield is in said first position.

5. The safety assembly of claim 1, wherein said latch extends integrally from said proximal end of said holder and is deflectable about an axis extending substantially parallel to an axis defined by the hinged connection of said shield to said holder.

6. The safety assembly of claim 1, wherein said latch extends integrally from said proximal end of said holder and is deflectable about an axis extending substantially orthogonal to an axis defined by the hinged connection of said shield to said holder.

7. The safety assembly of claim 1, wherein said latch is rotatably mounted to said holder for rotation about an axis substantially parallel to an axis defined by said tubular sidewall of said holder.

8. The safety assembly of claim 1, wherein said latch is movable along said holder substantially parallel to an axis defined by said tubular sidewall of said holder.

9. The safety assembly of claim 1, further comprising a piercing member mounted to said proximal end of said holder.

10. A safety assembly comprising:
a tube holder having a widely open proximal end, a partly closed distal end and a tubular sidewall extending between said ends;
a needle assembly mounted to said distal end of said holder and having a needle cannula with a sharply pointed distal end projecting distally beyond said holder;
a shield hingedly mounted to said proximal end of said holder for movement from a proximal position where said shield is substantially adjacent said tubular sidewall of said holder to a distal position where said shield shields portions of said needle cannula projecting distally from said holder;
a spring disposed for biasing said shield from said proximal position to said distal position; and
a latch disposed on said holder for movement between a locking position where said latch holds said shield in said proximal position and a release position where said latch permits said spring to propel said shield to said distal position.

11. The safety assembly of claim 10, wherein the shield include at least one lock for securely locking the shield in the distal position.

12. The safety assembly of claim 11, wherein the lock includes at least one resiliently deflectable cannula locking finger for locked engagement with said needle cannula when said shield is in said distal position.

13. The safety assembly of claim 10, wherein the shield in said proximal position extends substantially from said distal end of said holder to said proximal end thereof.

14. The safety assembly of claim 10, wherein said distal position of said shield is spaced from said proximal position of said shield by approximately 180°.

15. The safety assembly of claim 10, wherein said shield defines a substantially U-shaped channel having an open side facing away from said holder when said shield is in said proximal position.

16. The safety assembly of claim 10, wherein said latch extends integrally from said proximal end of said holder and is deflectable about an axis extending substantially parallel to an axis defined by the hinged connection of said shield to said holder.

17. The safety assembly of claim 10, wherein said latch extends integrally from said proximal end of said holder and is deflectable about an axis extending substantially orthogonal to an axis defined by the hinged connection of said shield to said holder.

18. The safety assembly of claim 10, wherein said latch is rotatably mounted to said holder for rotation about an axis substantially parallel to an axis defined by said tubular sidewall of said holder.

19. The safety assembly of claim 10, wherein said latch is movable along said holder substantially parallel to an axis defined by said tubular sidewall of said holder.
